(19) United States
(12) Patent Application Publication
(10) Pub. No.: US 2015/0211080 A1
(43) Pub. Date: Jul. 30, 2015

(54) MICROORGANISM DETECTING SYSTEM AND MICROORGANISM DETECTING METHOD

(71) Applicant: Azbil Corporation, Tokyo (JP)

(72) Inventor: Shinsuke YAMASAKI, Tokyo (JP)

(73) Assignee: AZBIL CORPORATION, Tokyo (JP)

(21) Appl. No.: 14/608,788

(22) Filed: Jan. 29, 2015

(30) Foreign Application Priority Data
Jan. 30, 2014 (JP) 2014-016120

(51) Int. Cl. C12Q 3/00

(52) U.S. Cl. CPC C12Q 3/00

(57) ABSTRACT

A microorganism detecting system includes: a microorganism detecting device that detects microorganisms included in the air through drawing in air and directing light into the air; at least one chamber that stores a culture medium for trapping microorganisms illuminated with light by the microorganism detecting device; and an opening/closing device that connects, when a microorganism is detected, or blocks, when no microorganism is detected, a path connecting the microorganism detecting device and the at least one chamber.

### CROSS REFERENCE TO RELATED APPLICATION

This application claims priority under 35 U.S.C. § 119 to Japanese Patent Application No. 2014-016120, filed on Jan. 30, 2014, the entire content of which being hereby incorporated herein by reference.

### FIELD OF TECHNOLOGY

The present disclosure relates to an environment evaluating technology and, in particular, relates to a microorganism detecting system and a microorganism detecting method.

### BACKGROUND

In clean rooms, such as bio clean rooms, airborne particles such as microorganisms are detected and recorded using particle detecting devices. See, for example, Japanese Unexamined Patent Application Publication No. 2011-83214, U.S. Pat. No. 8,358,411, and N. Hasegawa, et al., Instantaneous Bioaerosol Detection Technology and Its Application, azbil Technical Review, 2-7, Yamatake Corporation, December 2009. The state of wear of the air-conditioning equipment of the clean room can be ascertained from the result of the particle detection. Moreover, a record of particle detection within the clean room may be added as reference documentation to the products manufactured within the clean room. Optical particle detecting devices draw in air from a clean room, for example, and illuminate the drawn-in air with light. When microorganisms are included in the air, each of the individual microorganisms produces its autofluorescence, making it possible to detect the number of microorganisms that are included in the air through the frequency with which fluorescent light is detected.

Given this, an aspect of the present disclosure is to provide a highly reliable microorganism detecting system and microorganism detecting method.

### SUMMARY

One aspect of the present invention provides a microorganism detecting system including: (a) a microorganism detecting device that detects microorganisms included in the air through drawing in air and directing light into the air; (b) at least one chamber that stores a culture medium for trapping microorganisms illuminated with light by the microorganism detecting device; and (c) an opening/closing device, provided in the microorganism detecting device, which connects, when a microorganism is detected, or blocks, when no microorganism is detected, a path connecting an exhaust outlet through which air that has been illuminated with light is exhausted, and an injecting hole that is provided in at least one chamber.

Moreover, one aspect of the present invention provides a microorganism detecting method including: (a) detecting, by a microorganism detecting device, microorganisms included in the air through drawing in air and directing light into the air; and (b) connecting, when a microorganism is detected by the microorganism detecting device, or blocking, when no microorganism is detected, a path connecting an exhaust outlet through which air that has been illuminated with light is exhausted, provided in the microorganism detecting device, and an injecting hole that is provided in at least one chamber for containing a medium for trapping microorganisms.

The present invention enables the provision of a highly reliable microorganism detecting system and microorganism detecting method.

### BRIEF DESCRIPTIONS OF THE DRAWINGS

FIG. 1 is a schematic diagram of a microorganism detecting system as set forth in Example according to the present disclosure.

FIG. 2 is a schematic diagram of an optical microorganism detecting device as set forth in the Example according to the present disclosure.

FIG. 3 is a cross-sectional diagram of a light source element as set forth in the Example according to the present invention.

FIG. 4 is a schematic diagram of a chamber as set forth in the Example according to the present disclosure.

FIG. 5 is a schematic diagram of a chamber as set forth in the Example according to the present disclosure.

FIG. 6 is a schematic diagram of a chamber as set forth in the Example according to the present disclosure.

FIG. 7 is a schematic diagram of a microorganism detecting system as set forth in Another Example according to the present disclosure.

FIG. 8 is a schematic diagram of an optical microorganism detecting device according to yet another example according to the present disclosure.

### DETAILED DESCRIPTION

Examples of the present disclosure will be described below. In the descriptions of the drawings below, identical or similar components are indicated by identical or similar codes. Note that the diagrams are schematic. Consequently, specific measurements should be evaluated in light of the descriptions below. Furthermore, even within these drawings there may, of course, be portions having differing dimensional relationships and proportions.

### EXAMPLE

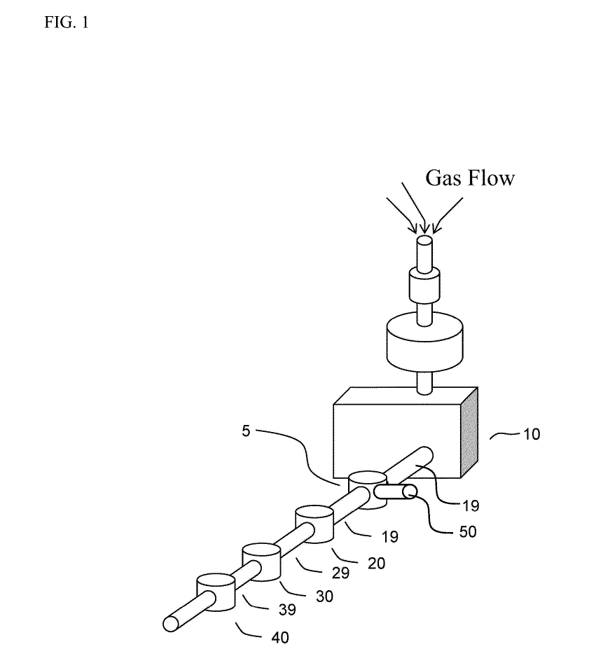

A microorganism detecting system according to Example, as illustrated in FIG. 1, comprises: a microorganism detecting device 10 for detecting microorganisms included in the air through drawing in air and directing light into the air; at least one chamber 20 for storing a culture medium for trapping microorganisms illuminated with light by the microorganism detecting device 10; and an opening/closing device 5, provided in the microorganism detecting device 10, for connecting, when a microorganism is detected, or for blocking, when no microorganism is detected, a path 19 connecting an exhaust outlet through which air that has been illuminated with light is exhausted, and an injecting hole that is provided in at least one chamber 20.

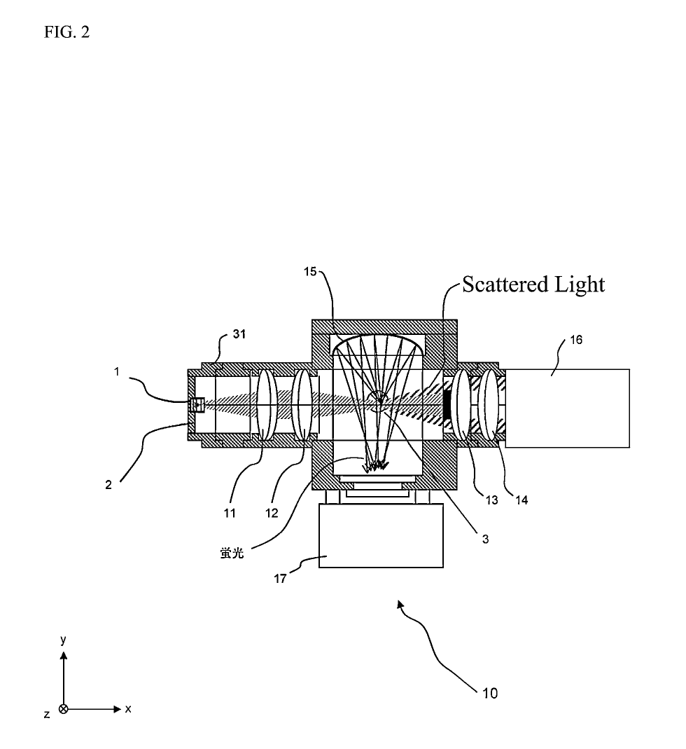

The optical microorganism detecting device 10 comprises: for example, as illustrated in FIG. 2, a light source element for emitting light; a base 2 on which the light source element 1 is installed; an emission-side collimating lens 11 for collimating light emitted from the light source element 1; an emission-side focusing lens 12 for focusing the collimated light; and a nozzle mechanism 3 for causing a gas flow, which includes microorganisms, to cross the beam that is focused by the emission-side focusing lens 12. The nozzle mechanism 3 may comprise an air valve for changing the flow rate of the gas flow, for example

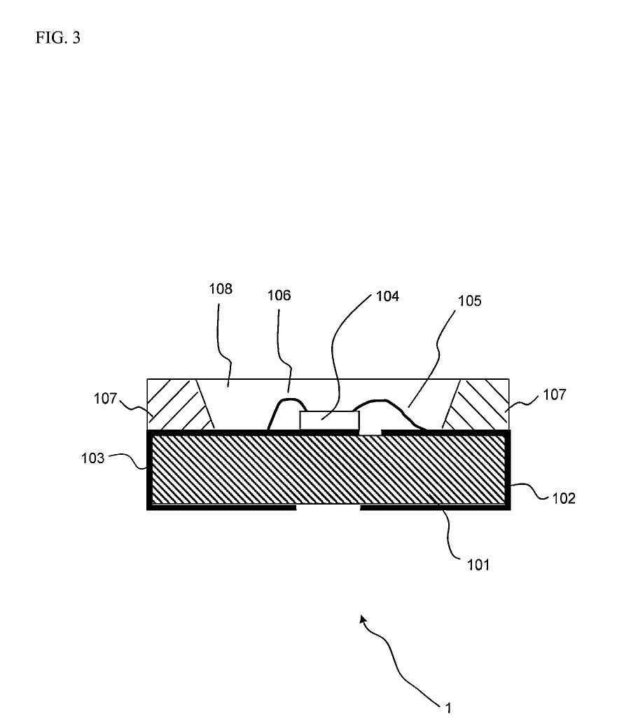

The light source element 1 that is installed on the base 2, as illustrated in FIG. 3, for example, comprises: a substrate 101; an anode electrode 102 that is disposed along the surface of the substrate 101; a cathode electrode 103; and a light-emitting diode (LED) chip 104 that is disposed on top of the substrate 101. The anode electrode 102 and the LED chip 104 are connected electrically through wire bonding 105. Moreover, the cathode electrode 103 and the LED chip 104 are connected electrically through wire bonding 106. A reflector 107 is disposed on top of the substrate 101 so as to surround the LED chip 104. Moreover, the LED chip 104 is encapsulated in transparent resin 108.

The light that is emitted from the light source element 1 may be visible light or may be ultraviolet light. In the case of the light being visible light, the wavelength of the light is in the range of for example, between 400 and 410 nm, for example, 405 nm. In the case of the light being ultraviolet light, the wavelength of the light is in the range of, for example, between 310 and 380 nm, for example, 355 nm. Note that the wavelength of the light emitted from the light source element 1 is determined by the type of microorganism that is to be detected. The base 2 for holding the light source element 1, illustrated in FIG. 2, is secured to a case 31 of the optical microorganism detecting device 10.

The nozzle mechanism 3 draws in air from the outside of the case 31, using a fan, or the like, and then emits a nozzle of the air that has been drawn in in the direction of the focal point of the emission-side condensing lens 12. The direction in which the airstream that is jetted from the nozzle mechanism 3, relative to the direction of propagation of the light condensed by the emission-side condensing lens 12 is set to, for example, essentially perpendicular. If a microorganism is included in the air here, then the light that strikes the microorganism is scattered through Mie scattering, producing scattered light. Furthermore, the nicotinamide adenine dinucleotide (NADH) and the flavins, and the like, that are included in in microorganisms that are illuminated with light produce fluorescent light. Note that the air need not necessarily be blown in the direction of the focal point of the emission-side focusing lens 12. For example, insofar as the air crosses the beam, it may be blown to a position other than the focal point of the emission-side focusing lens 12.

Examples of microorganisms include bacteria and fungi. Examples of such microbes include Gram-negative bacteria, Gram-positive bacteria, and fungi such as mold spores. Escherichia coli, for example, can be listed as an example of a Gram-negative bacterium. Staphylococcus epidermidis, Bacillus atrophaeus, Micrococcus lylae, and Corynebacterium afermentans can be listed as examples of Gram-positive bacteria. Aspergillus niger can be listed as an example of a fungus such as a mold spore. The airstream the cuts across the light that is condensed by the emission-side condensing lens 12 is exhausted into a path 19, such as a pipe as illustrated in FIG. 1, from an exhaust opening that is provided in the case 31 by an exhausting mechanism.

The optical microorganism detecting device 10 illustrated in FIG. 2 further comprises a detecting-side collimating lens 13 for forming into a collimated beam the light that was cut-across by the airstream jetted by the nozzle mechanism 3, and a detecting-side condensing lens 14 for condensing the beam that was collimated by the detecting-side collimating lens 13. When scattered light is produced through a microorganism included in the airstream, the scattered light is also collimated by the detecting-side collimating lens, and thereafter is condensed by the detecting-side condensing lens 14.

A scattered light detecting portion 16 for detecting light scattered by microorganisms is disposed at the focal point of the detecting-side condensing lens 14. The scattered light detecting portion 16 may use, for example, a photodiode, a photoelectron multiplier tube, or the like. The scattered light detecting device is able to count the number of microorganisms from the number of times that scattered light is detected by the scattered light detecting portion 16. Moreover, the intensity of the light that is scattered from the microorganisms is correlated to the diameters of the microorganisms. Consequently, detecting the intensity of the scattered light using the scattered light detecting portion 16 makes it possible to calculate the size of the airborne microorganisms in the environment wherein the optical microorganism detecting device 10 is placed.

A condensing mirror 15, which is a concave mirror, is also placed within the case 31 of the optical microorganism detecting device 10 in parallel with the airstream that is jetted from the nozzle mechanism 3. The condensing mirror 15 condenses the florescent light that is emitted from microorganisms included within the airstream. A florescent light detecting portion 17, for detecting the florescent light, is disposed at the focal point of the condensing mirror 15. When scattered light is detected by the scattered light detecting portion 16 and florescent light is detected by the florescent light detecting portion 17 as well, then it is understood that the particle included in the airstream is a microbe particle. When scattered light is detected by the scattered light detecting portion 16 and florescent light is detected by the florescent light detecting portion 17 as well, then it is understood that the particle included in the airstream is a microbe particle such as a microorganism. Moreover, the fluorescent light detecting device is able to count the number of microorganisms from the number of times that fluorescent light is detected by the scattered light detecting portion 17. A computer for performing statistical processes in real-time on the light intensities and florescent light intensities that are detected is connected to the scattered light detecting portion 16 and the florescent light detecting portion 17. The opening/closing device 5 illustrated in FIG. 1 is connected to a computer.

The opening/closing device 5 is provided with a valve, or the like. The opening/closing device 5 is provided in the microorganism detecting device 10 and connects a path 19 that connects an exhaust outlet, for exhausting air that has been illuminated with light, and an injecting hole that is provided in at least one chamber 20 only when the microorganism detecting device 10 has detected fluorescent light that has been emitted from an organism particle. When the microorganism detecting device 10 has not detected fluorescent light emitted from an organism particle, the opening/closing device 5 blocks the path 19. As a result, the air that has been illuminated with light in the optical microorganism detecting device 10 passes through the path 19 to be sent to the chamber 20 only when an organism particle has been detected. When an organism particle has not been detected, the air that is illuminated with the light in the optical microorganism detecting device 10 is directed to an exhaust path 50 through an opening/closing device 5, to be exhausted to the outside.

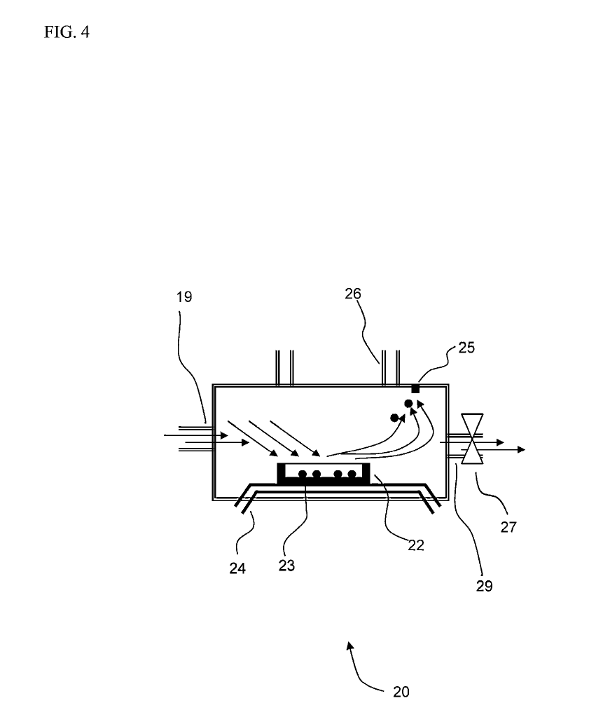

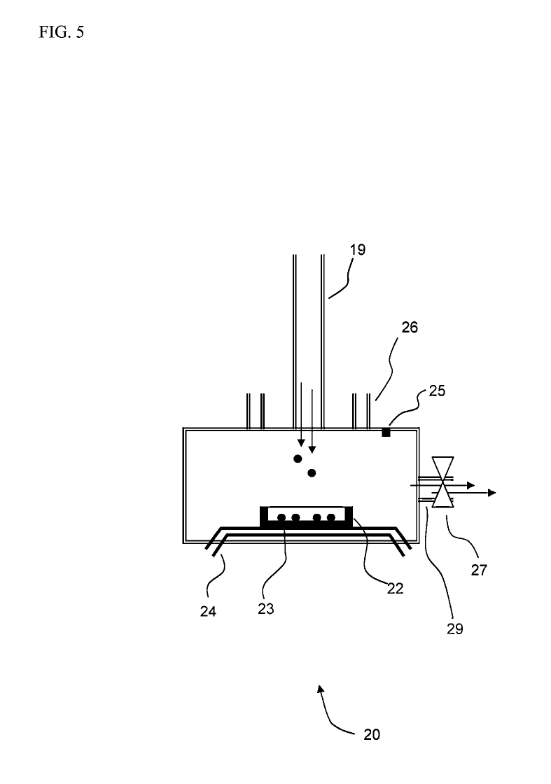

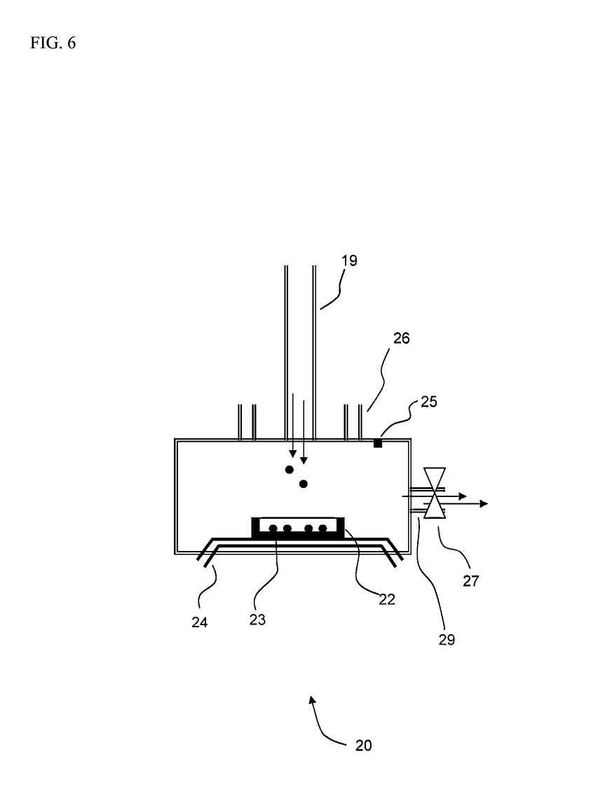

As illustrated in FIG. 4, the chamber 20 contains, for example, a petri dish 22. A culture medium 23 is filled into the Petri dish 22. At least some of the microorganisms that are included in the air that is inspected in the optical microorganism detecting device 10 adhere to the culture medium 23, to be cultured on the culture medium 23. Note that, as illustrated in FIG. 5, the path 19 and the chamber 20 may be connected so that the culture medium 23 will be perpendicular to the direction in which the gas that contains the microorganism flows. Moreover, as illustrated in FIG. 6, a nozzle 28 may be provided on the opening of the path 19, depending on the size of the microparticles that are to be trapped. When a microorganism that is included in the gas flow strikes the culture medium 23, culturing of the microorganism in the culture medium 23 commences immediately, relieving the microorganisms of the stress of being dry, the stress of inadequate nutrition, and the like. The microorganisms cultured on the culture medium 23 are observed visually, or are dyed as necessary and observe through an optical microscope, or the like.

The microorganism detecting system according to the Example may further comprise a temperature controlling device for controlling the temperature within the chamber 20. The temperature controlling device comprises a temperature adjusting pipe 24 for supplying a coolant therein, for example. Conversely, the temperature controlling device may comprise a Peltier element.

Moreover, the microorganism detecting system according to the Example may further comprise a humidity controlling device for controlling the humidity within the chamber 20. The humidity controlling device may, for example, comprise a humidity sensor 25 and a dry gas flow supply pipe 26. The humidity sensor 25 detects the humidity within the chamber 20. If the value of the humidity detected by the humidity sensor 25 is higher than a prescribed value, then dry air is supplied into the chamber 20 from the dry gas flow supply pipe 26, to control the humidity of the chamber 20. While typically bacteria proliferate on foodstuffs with high moisture activity, yeast proliferates on foodstuffs with relatively low moisture activity, and molds proliferate on foodstuffs with even lower moisture activity. However, halophilic bacteria proliferate even when the moisture activity is extremely low, and drought-resistant molds and osmotolerant yeasts can grow with even lower moisture activity. Consequently, when microorganisms that can proliferate with low moisture activity are to be detected, then the chamber 20 may be dehumidified through the humidity controlling device.

The air in the chamber 20 is drawn, by a suction device, through the pipe 29, the filtering device 30, and the pipe 29, illustrated in FIG. 1. As illustrated in FIG. 4, a valve 27 may be provided on the suction device 40. The filtering device 30, illustrated in FIG. 1 is provided with, for example, a HEPA (High-Efficiency Particulate Air) filter, to prevent microorganisms, and the like, from being exhausted into the atmosphere through the suction device 40. A pump, or the like, may be used as the suction device 40.

Conventionally, the air that has been inspected by the optical microorganism detecting device is filtered constantly by a gelatinous filter, and the microorganisms trapped by the gelatinous filter are cultured. However, in the conventional method there are cases wherein there is no correlation between the number of microorganisms detected by the optical microorganism detecting device and the number of microorganism colonies trapped and cultured by the gelatinous filter. After diligent research, the present inventor discovered that, due to exposure to the exhaust of the optical microorganism detecting device, the gelatinous filter becomes dry, so the microorganisms trapped in the gelatinous filter do not survive. Moreover, the present inventor discovered that there are cases wherein the microorganisms trapped in the gelatinous filter die due to a lack of nutrition.

In contrast, the microorganism detecting system according to the Example according to the present invention comprises an opening/closing device 5, provided in the microorganism detecting device 10, for connecting, when a microorganism is detected, and for blocking, when no microorganism is detected, a path 19 that connects an exhaust outlet for exhausting air that has been illuminated with light and an injecting hole that is provided in at least one chamber 20, so that the air that has been inspected by the optical microorganism detecting device 10 does not constantly blow against the culture medium 23, illustrated in FIG. 4 through FIG. 6, within the chamber 20. This suppresses drying of the culture medium 23, making it possible to suppress death, due to drying, of the microorganisms trapped in the culture medium 23. Moreover, if the culture medium 23 includes nutrients, this can suppress death of the microorganisms due to a lack of nutrition. Consequently, when the number of microorganisms detected by the microorganism detecting device 10 is compared to the number of microorganism colonies that are trapped and cultured in the culture medium 23, there is likely to be a correlation.

### ANOTHER EXAMPLE

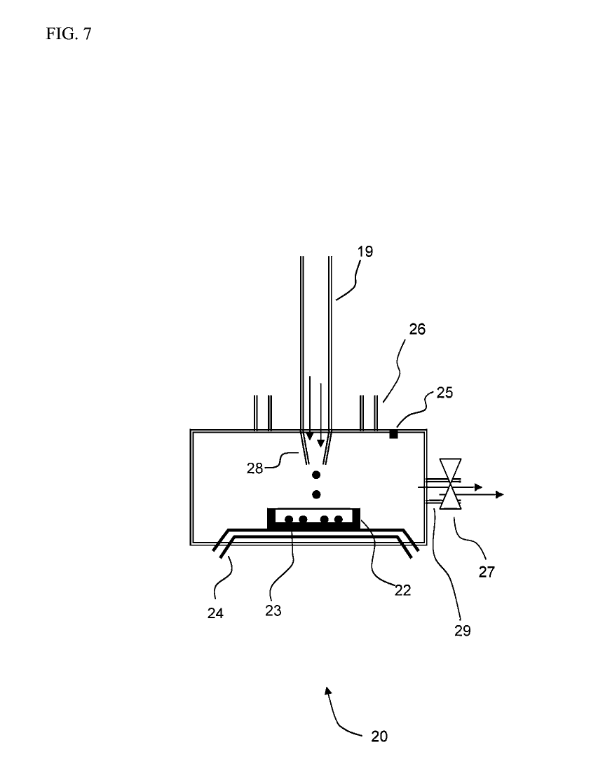

A microorganism detecting system according to the Another Example is provided with a plurality of chambers 20A and 20B, as illustrated in FIG. 7, where a plurality of opening/closing devices 5A and 5B sort, into the plurality of chambers 20A and 20B, the microorganisms detected by the microorganism detecting device 10 depending on the characteristics of the microorganisms, such as particle size, and the like. As described above, the intensity of the light scattered by the microorganisms is correlated to the particle sizes of the microorganisms, enabling the optical microorganism detecting device 10 to determine the sizes of the detected microorganisms. Here, if, for example, the microorganism is a bacterium, the particle size of the bacterium is, for example, between 0.5 and 1.0 μm. Moreover if the microorganism is a fungus, the particle size of the fungus is, for example, between 1.0 and 5.0 μm. As a result, a microorganism can be identified as a bacterium versus a fungus based on the size of the microorganism that is detected.

Bacteria and fungi have different culturing conditions. For example, when culturing a bacterium, tryptose agar (TSA) is used as the culture medium, and the temperature is set to 32° C. When culturing a fungus, for example, potato dextrose agar (PDA) is used as the culturing medium, and the temperature is set to 25° C.

In the Another Example, the exhaust duct for the optical microorganism detecting device 10 is connected to the opening/closing devices 5A through a path 19. Path 19A and path 19C are connected to the opening/closing device 5A. A chamber 20A is connected to the path 19A. The interior of the chamber 20A is set to an environment that is suitable for culturing bacteria, with a TSA culture medium disposed therein. The opening/closing device 5B is connected to the path 19C. The path 19B and an exhaust path 50 are connected to the opening/closing device 5B. A chamber 20B is connected to the path 19B. The interior of the chamber 20B is set to an environment that is suitable for culturing fungus, with a PDA culture medium disposed therein.

When a fluorescent particle of a size corresponding to a bacterium is detected by the optical microorganism detecting device 10, the opening/closing device 5A connects the optical microorganism detecting device 10 to the chamber 20A. Moreover when a fluorescent particle of a size corresponding to a fungus is detected by the optical microorganism detecting device 10, the opening/closing devices 5A and 5B connects the optical microorganism detecting device 10 to the chamber 20B. When neither a bacterium nor a fungus is detected by the optical microorganism detecting device 10, then the opening/closing devices 5A and 5B connect the optical microorganism detecting device 10 and the exhaust path 50. The air within the chamber 20A is drawn, by the suction device 40A, through a pipe 29A, a filtering device 30A, and a pipe 39A. The air within the chamber 20B is drawn, by the suction device 40B, through a pipe 29B, a filtering device 30B, and a pipe 39B.

In the microorganism detecting system according to the Another Example, when multiple types of microorganisms are included in the air that is to be inspected, it is possible to perform culturing that is suited to each individual type of microorganism. The microorganism detecting system may be provided with three or more chambers and three or more opening/closing devices.

### OTHER EXAMPLES

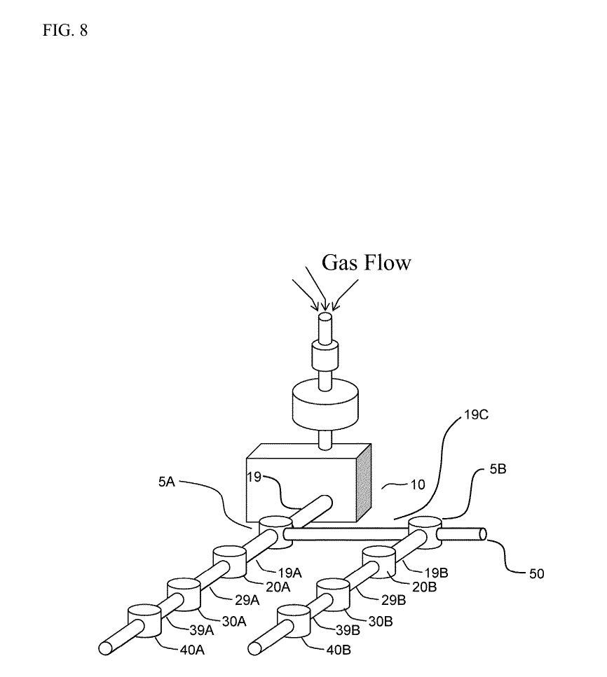

While there are descriptions of examples as set forth above, the descriptions and drawings that form a portion of the disclosure are not to be understood to limit the present disclosure. A variety of alternate examples and operating technologies should be obvious to those skilled in the art. For example, the optics system in the optical microorganism detecting device 10 is not limited to the example illustrated in FIG. 2. For example, as illustrated in FIG. 8, the gas flow that includes the microorganisms may cross a beam that is collimated by the collimating lens 51. In this way, the present disclosure should be understood to include a variety of examples, and the like, not set forth herein.

1. A microorganism detecting system comprising:
a microorganism detecting device that detects microorganisms included in the air through drawing in air and directing light into the air;
at least one chamber that stores a culture medium for trapping microorganisms illuminated with light by the microorganism detecting device; and
an opening/closing device that connects, when a microorganism is detected, or blocks, when no microorganism is detected, a path connecting the microorganism detecting device and the at least one chamber.

2. The microorganism detecting system as set forth in claim 1, wherein:
a microorganism that has been included in air exhausted from the microorganism detecting device is cultured by a culture medium.

3. The microorganism detecting system as set forth in claim 1, wherein:
a plurality of chambers are provided; and
the opening/closing device sorts, into the plurality of chambers, microorganisms depending on microorganism characteristics detected by the microorganism detecting device.

4. The microorganism detecting system as set forth in claim 1, further comprising:
a temperature controlling device that controls a temperature in the at least one chamber.

5. The microorganism detecting system as set forth in claim 1, further comprising:
a humidity controlling device for controlling a humidity in the at least one chamber.

6. A microorganism detecting method comprising:
a detecting step of detecting, by a microorganism detecting device, microorganisms included in the air through drawing in air and directing light into the air; and
a opening/closing step of connecting when a microorganism is detected, or blocking when no microorganism is detected, by an opening/closing device, a path connecting the microorganism detecting device and the at least one chamber.

7. The microorganism detecting method as set forth in claim 6, further comprising:
a step of culturing, by a culture medium, a microorganism that has been included in air exhausted from the microorganism detecting device.

8. The microorganism detecting method as set forth in claim 7, wherein:
the number of microorganisms detected by the microorganism detecting device is compared to the number of microorganisms cultured in the culture medium.

9. The microorganism detecting method as set forth in claim 8, wherein:
a number of microorganisms cultured in the culture medium is a number of colonies of the microorganisms

10. The microorganism detecting method as set forth in claim 6, wherein:
a plurality of chambers are provided; and
microorganisms are sorted into a plurality of chambers, depending on microorganism characteristics detected by the microorganism detecting device.

11. The microorganism detecting method as set forth in claim 6, further comprising:
a step of controlling a temperature in the at least one chamber.

12. The microorganism detecting method as set forth in claim 6, further comprising:
a step of controlling a humidity in the at least one chamber.
